(19) United States
(12) Patent Application Publication
(10) Pub. No.: US 2019/0117974 A1
(43) Pub. Date: Apr. 25, 2019

(54) TOPICAL NERVE STIMULATION DEVICE

(71) Applicant: The Procter & Gamble Company, Cincinnati, OH (US)

(72) Inventors: Graham Harold CREASEY, Menlo Park, CA (US); Noel GAUTHIER, Cincinnati, OH (US); Erik John HASENOEHRL, Loveland, OH (US); Jeanne Marie HUGHES, Loveland, OH (US); Hoo-min D. TOONG, Cambridge, MA (US)

(21) Appl. No.: 16/165,293

(22) Filed: Oct. 19, 2018

Related U.S. Application Data
(60) Provisional application No. 62/574,625, filed on Oct. 19, 2017.

(51) Int. Cl. A61N 1/36; A61N 1/04

(52) U.S. Cl. CPC A61N 1/36014; A61N 1/0492; A61N 1/0456

(57) ABSTRACT

The present disclosure is directed to a transcutaneous nerve stimulation device and system for treating overactive bladder (OAB) and its symptoms. The devices and systems described herein are intuitively shaped to enable proper placement by the user to stimulate the user's tibial nerve.

### FIELD OF THE INVENTION

The present disclosure is directed to a transcutaneous nerve stimulation device and system for treating overactive bladder (OAB) and its symptoms. The devices and systems described herein are intuitively shaped to enable proper placement by the user to stimulate the user's tibial nerve.

### BACKGROUND OF THE INVENTION

Overactive bladder (OAB) is a common condition that affects millions of Americans. As many as 30% of men and 40% of women in the United States live with OAB symptoms. The most common symptom of OAB is a sudden urge to urinate that one cannot control (sometimes referred to as urge incontinence), which can lead to wetting accidents. Having to go to the restroom many times during the day is common for people suffering from OAB. And, the number of people suffering from OAB is likely larger than is thought, because many people living with OAB do not talk to their health care provider about the condition or ask for help to treat the condition. Some are embarrassed by the condition and others do not discuss the condition or ask for help because they believe there are not any treatments for OAB.

Management of OAB often begins with behavioral strategies, such as fluid schedules, timed voiding, and bladder-holding techniques using a person's pelvic floor. If these behavioral strategies do not help enough, medications are available. However, many people have reservations about taking medications and medications may have unwanted side effects. Another treatment for OAB requires the surgical implantation of a nerve stimulator by a doctor or, for women, an electrical stimulation treatment may be administered via an intravaginal probe or may be worn intravaginally or perineally. These treatments can be difficult to administer or uncomfortable for the user, particularly when used for a long period of time.

Thus, there is a need for an OAB treatment that is non-invasive, effective, can be applied by the user by herself or himself (without assistance from another person, including a medical professional), is less obtrusive, and is available without a prescription.

### SUMMARY OF THE INVENTION

The present disclosure relates to a topical nerve stimulation device for treating overactive bladder in a human user, the device comprising: at least two electrode configured to contact a surface of the medial ankle; a power source in electrical communication with the electrodes; wherein the device is shaped and configured for placement proximate to the medial malleolus to stimulate the tibial nerve.

The present disclosure also relates to a topical nerve stimulation system for treating overactive bladder in a human user, the system comprising: (a) a body-facing layer; (b) a garment-facing layer; (c) at least two electrodes, the electrodes configured to contact a surface of the medial ankle, and a power source in electrical communication with the electrodes, the power source disposed intermediate the garment-facing layer and the body-facing layer; wherein the system is shaped and configured for placement proximate to the medial malleolus to stimulate the tibial nerve.

The present disclosure also relates to methods of treating overactive bladder and/or its symptoms comprising a) providing the device or system disclosed herein to a user experiencing a symptom of overactive bladder; b) affixing the device of claim 1 to one of the user's medial ankles; c) activating or stimulating the tibial nerve of the user.

### BRIEF DESCRIPTION OF THE DRAWINGS

FIG. 1 is a medial view of the anatomy of an ankle.

FIG. 2 is a block diagram of a nerve activation device in accordance with the disclosure.

FIG. 3 is a block diagram of a nerve activation device in accordance with the disclosure.

FIGS. 4a-4g. shows stimulation systems affixed to ankles in accordance with the disclosure.

FIGS. 5a-b. shows stimulation system affixed to an ankle in accordance with the disclosure.

FIGS. 6a-6g. show comparative examples of stimulation systems affixed to ankles.

FIG. 7. shows a comparative example of a stimulation system affixed to an ankle.

FIG. 8. is an exploded view of an stimulation system in accordance with the disclosure.

FIG. 9 is a top perspective view of the system of FIG. 6.

FIG. 10 is a cross-sectional view of the layers of the system of FIG. 6.

### DETAILED DESCRIPTION OF THE INVENTION

The terms “stimulate” and “activate” will be used interchangeably throughout the application.

It will be understood that when an element is referred to as being “connected to” or “coupled to” or “in electrical communication with” to another element, it can be directly connected to, coupled to, or in electrical communication with the other element or intervening elements may be present. In contrast, if an element is referred to as being “directly connected” or “directly coupled” or “in direct electrical communication with” to another element, there are no intervening elements present.

To facilitate understanding, like or identical reference numerals have been used, where possible, to designate like or identical elements common to the figures.

The disclosure relates to an electrical stimulation or activation device and a related electrical stimulation or activation system for treatment of over active bladder (OAB) and its symptoms in a human user via transcutaneous electrical nerve stimulation. The devices described herein are small, light, and intuitively shaped to enable proper placement by the user to stimulate the user's tibial nerve 300. Advantages of the devices over existing transcutaneous electrical nerve stimulation devices are: easy and intuitive for the user to apply, without the assistance of a medical professional or other person; require less power to operate; lower overall profile and a smaller footprint over a surface, such as a user's skin surface; more compact; less obtrusive; discrete; fewer parts and easier to manufacture or assemble; integrated power, communications, stimulating, and optionally sensing; inexpensive; disposable; convenient; no wires to become entangled in clothing; showerproof and sweat proof; allows for control of stimulation parameters from a remote device such as a smartphone, either directly by the user or by stored programs. The devices described herein effectively activate a target nerve(s), while not affecting untargeted nerves. The devices described herein are expected to have service lifetimes of days to weeks and their disposability places less demand on power sources and battery requirements.

### Nerve Stimulation Device

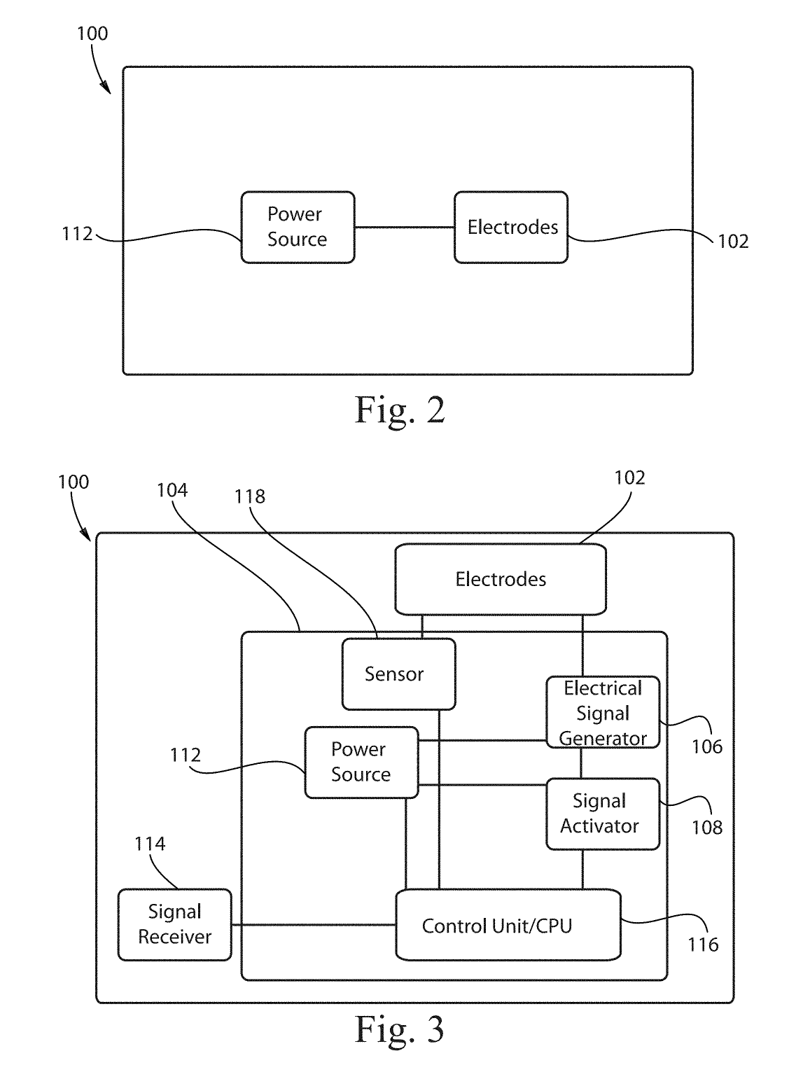

Reference is now made to FIGS. 2 and 3, which are schematic block diagrams of transcutaneous nerve stimulation devices for treating overactive bladder (OAB) and its symptoms. As seen in FIG. 2, the device 100 for treating OAB and its symptoms may include electrodes 102 and a power source 112 in electrical communication with the electrodes 102. When the electrodes 102 are in electrical communication with the power source 112, the electrodes 102 continuously draw energy from the power source 112 and generate an electric field of suitable strength to stimulate the target nerve. As seen in FIG. 3, the device may optionally include an electrical signal generator 106 in electrical communication with the electrodes 102, where the electrical signal generator 106 generates an electrical signal adapted to stimulate the target nerve. When the electrodes 102 receive the signal from the generator 106, they draw energy from the power source 112 and generate an electric field of suitable strength to stimulate the target nerve.

As seen in FIG. 3, the device may optionally include a signal activator 108 coupled to or in electrical communication with the electrical signal generator 106, where the electrical signal generator 106 receives instructions from the signal activator 108 and where the electrical signal generator 106 and the signal activator 108 are in electrical communication with the power source 112. The signal activator 108 may be a button on the device or, in the case of a wireless device, a fob.

The device may also include an amplitude modulator (not shown) in electrical communication with the electrical signal generator, such as the modulator having the designation On-Semi MC1496, which is sold by Texas Instruments. The modulator generates a modulated waveform that is transmitted to the electrodes 102, which in turn apply the modulated waveform to the target nerve.

As seen in FIG. 3, the device may optionally include a control unit or CPU 116, which may perform functions, such as, data processing, communications, and storage. The control unit 116 may run software that controls the local functions of the device. The control unit 116 may be a cellular telephone, a laptop, a tablet, a dedicated hardware device, such as a key fob, or some other handheld device. The device may also optionally include a signal receiver (e.g., antenna) 114, for wireless external communication, and/or one or more sensors 118, such as, but not limited to, mechanical motion and pressure, temperature, humidity, chemical and positioning sensors. In particular, the sensor 118 may detect the volume or pressure in the bladder. The device may also include a light emitting element (not shown), such as an LED, for generating light signals indicating that the device is turned on. The electrical components of the device, excluding the electrodes, may be referred to collectively as the “electrical components.” The electrical components may be mounted on substrate, such as a circuitized substrate, e.g., a printed circuit board.

The electrodes 102 of the device are applied to the skin and provide electrical stimuli through the skin to the tibial nerve 300. At least one electrode is an anode and at least one electrode is a cathode, with current flowing from an anode to a cathode. Stimuli may typically be trains of voltage-regulated square waves at frequencies between 1 and 150 Hz with currents between 20 and 100 mA. Stimuli may be either initiated by the user when desired, or programmed according to a timed schedule, or optionally initiated in response to an event detected by a sensor 118 that monitors some biometric of the user. The electrodes 102 may optionally collect electrical signals from the body to provide data about body functions.

Suitable electrodes 102 include both dry and floating electrodes. Dry electrodes are in direct contact with skin, where as floating electrodes use an electrolytic gel as a chemical interface between the electrode and skin. The electrolytic gel may form reliable electrical interconnections between the electrodes 102 and a user's skin and may help affix the device to the surface of a user's skin, e.g., self-adhesive hydrogel electrodes. Electrodes 102 can comprise metal, conductive polymers, electrically conductive thin films, conductive carbon. Electrolytic gels are typically hydrogels. One such electrode is commercially available as Sticky Pad™ Surface electrode sold by Rhythmlink® TENS Pros Premium® TENS Stim Electrodes is another example. The hydrogel may be as thin as possible, in order to lower the profile of the device.

Each electrode 102 may have a body-facing surface area of from about 500 mm² to about 1100 mm². The electrodes 102 may have different or identical body-facing surface areas. The sum of the body-facing surface areas of the electrodes 102 may be less than 3,000 mm², preferably from about 1050 mm² to about 2200 mm², more preferably from about 500 mm² to 2200 mm².

The electrodes 102 may be spaced about 1 mm apart to about 100 mm apart (edge to edge), preferably about 5 mm apart to about 80 mm apart, more preferably about 10 mm apart to about 60 mm apart. The distance between the electrodes 102 may be selected to maximize the effectiveness of the electrical signal at the target nerve and/or minimize the footprint of the nerve stimulation system (as described above).

Table 1 shows the number of pulses per treatment measured against two parameters, frequency and duration. Frequency is shown on the Y-axis and duration on the X-axis. Referring to Table 1, a frequency setting of 20 Hz and duration of 10 seconds produces 200 pulses.

TABLE 1

| Frequency | Duration (seconds) | | | | | |
|---|---|---|---|---|---|---|
| (Hertz) | 1 | 5 | 10 | 20 | 30 | 60 |
| 1 | 1 | 5 | 10 | 20 | 30 | 60 |
| 5 | 5 | 25 | 50 | 100 | 150 | 300 |
| 20 | 20 | 100 | 200 | 400 | 600 | 1200 |
| 50 | 50 | 250 | 500 | 1000 | 1500 | 3000 |
| 100 | 100 | 500 | 1000 | 2000 | 3000 | 6000 |
| 150 | 150 | 750 | 1500 | 3000 | 4500 | 9000 |

The power source 112 may be a low voltage power source, providing a nominal voltage of at most 10.0 volts, at most 8.0 volts, at most 6.0 volts, at most 5.0 volts, at most 4.0 volts, or at most 3.0 volts. The power source 112 may be a battery. Batteries of different shapes and sizes may be used, where the shape and size of the battery may be selected based, at least in part, on the shape and size of the topical nerve stimulation device. Suitable batteries include alkaline batteries, silver batteries, zinc-air batteries, lithium ion batteries, lithium polymer batteries, nickel oxyhydroxide batteries, and mercury batteries. The batteries may be printable batteries, button batteries, or any other common batteries. The battery may be rechargeable.

The battery may have a capacity of less than about 1000 milliampere hours (mAh) or from about 1 mAh to about 1000 mAh, preferably from about 1 mAh to about 500 mAh, more preferably about 1 mAh to about 100 mAh, even more preferably about 1 mAh to about 50 mAh.

### Electrical Signal Generator

The electrical signal generator (also referred to as an electrical pulse generator) generates an electrical signal, preferably an electrical signal configured or adapated to stimulate a target nerve. When the electrodes 102 receive the signal from the generator, the electrodes 102 draw energy from the power source 112, e.g., battery, and generate an electrical field of suitable strength to stimulate the target nerve. The electrical field may intersect or overlap the target nerve. The electric field may activate the nerve by triggering its action potential, causing the nerve to send signals along its pathway. The electrical signal generator may be of any suitable type, such as those sold by Texas Instruments of Dallas, Tex. under model number NE555.

### Signal Activator

The signal activator may be coupled with the power source 112 for turning on the device. The signal activator may be a single-use activator that is configured to be turned on only one time. The signal activator may be a switch, such as a push button switch.

### Nerve Stimulation System

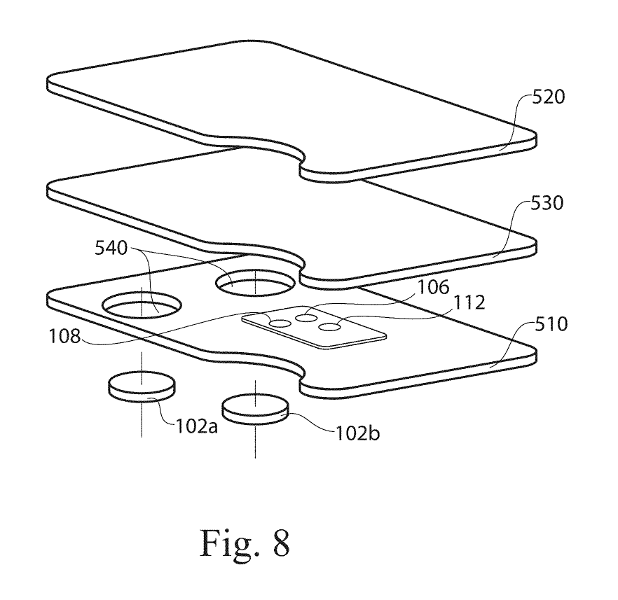

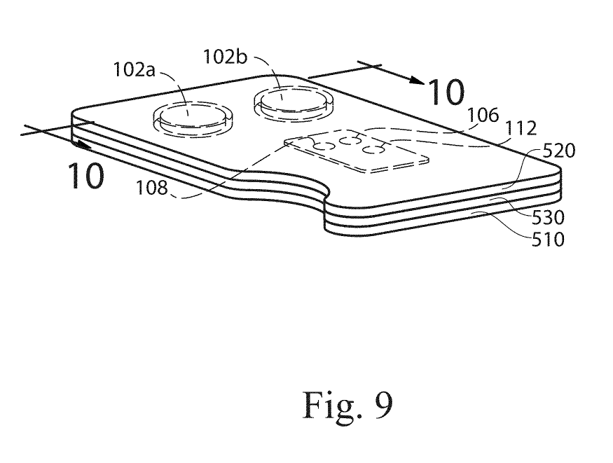

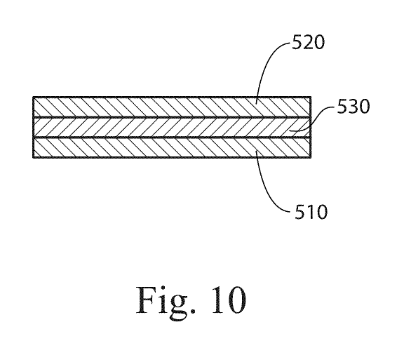

The electrodes 102 and the electrical components of the nerve stimulation device, as described herein, may be combined or assembled with any suitable materials to create a nerve stimulation system. Minimally, the system may consist of or consist essentially of the electrodes 102 and the selected electrical components (in appropriate electrical communication, as described above) and few, if any, other materials, for example, the electrodes in electrical communication with the power source 112, where the electrodes 102 and power source 112 are mounted on a film that is cut to fit the electrodes 102 and the power source 112. FIG. 8 illustrates an outside perspective view of a nerve stimulation system 500. The system 500 may include an exterior surface (garment-facing or non-body-facing) formed by a garment-facing 520 or non-body-facing layer 520 sometimes referred to as the garment-facing layer 520, and an interior surface (body-facing) formed by a body-facing layer 510 sometimes referred to as the body-facing layer 510 or adhesive layer 510. The body-facing layer 510 includes adhesive.

The electrodes 102a, 102b may be integrated in the body-facing layer. The body-facing layer 510 may include openings 540 extending therethrough that accommodate the electrodes 102a, 102b and allow direct contact of the electrodes with the surface of the user's skin. Integrating the electrodes into the body-facing layer 510 may minimize the size and footprint of the system, e.g., lower overall profile and/or smaller footprint over the surface of the user's skin. The electrodes 102a, 102b may be held in place by the garment-facing 520 or non-body-facing 520 layer. The electrodes may be arranged in a variety of configurations. The distance between the electrodes may be selected to maximize the effectiveness of the electrical signal at the target nerve and/or minimize the footprint of the nerve stimulation system (as described above).

The electrical components, including the power source 112, the optional electrical signal generator 106, the optional signal activator 108, and any other optional components as described herein, are disposed intermediate the body-facing layer 510 and the garment-facing layer 520 layer. The other electrical components may be arranged in any configuration, provided that the optional electrical signal generator is in electrical communication with the electrodes, the optional signal activator is coupled to or in electrical communication with the optional electrical signal generator, and the power source 112 is in electrical communication with the optional electrical signal generator and the optional signal activator.

The body-facing layer 510 may be a layer of one or more materials that forms at least a portion of the inside of the system and faces a wearer's skin when the system 500 is worn by the wearer. A body-facing layer is sometimes referred to as an adhesive layer. The body-facing layer 510 may be configured to be flexible and to carry the adhesive. A body-facing layer 510 may include a woven material, a nonwoven material, plastic material, latex material, hydrogel material, hydrocolloid material, and/or other materials as long as the materials are suitable for carrying adhesive.

The garment-facing or non-body-facing layer 520 may be a layer formed of one or more materials that form at least a portion of an outside of the system 500 and may face a wearer's garments, e.g., pants, socks, when the system 500 is worn by the wearer. A garment-facing layer 520 is sometimes referred to as a backsheet or backing layer. The garment-facing layer 520 may be configured to be flexible and may be liquid impermeable or may be breathable. A garment-facing layer 520 may include plastic material (injection molded, blow molded, thermoformed etc.), coated or uncoated paper, a nonporous film, a porous film, a woven material, a non-woven fibrous material or combinations thereof. The outer cover may also be stretchable, extensible, elastically extensible, or elastomeric. The garment-facing layer 520 may also be vapor permeable and yet liquid impervious.

The system can also be made more comfortable by the addition of material between the garment-facing layer 520 and the body-facing layer 510, such as cushioning material 530 that can cushion the electrodes 102a, 102b and electronic components. The cushioning material 530 may be disposed subjacent to the body-facing layer 510 and superjacent to the garment-facing layer 520, in at least a portion of the system 500. A cushioning material may include cellulosic fibers (e.g., wood pulp fibers), other natural fibers, synthetic fibers, woven or nonwoven sheets, scrim netting or other stabilizing structures, superabsorbent material, foams, binder materials, or the like, as well as combinations thereof.

### Body-Facing Layer (Adhesive Layer)

Any conventional adhesive layer materials may be used within the device system. Suitable adhesive layers may be made from plastic materials, nonwoven materials, silicone, acrylic, hydrogel, or latex, including polyvinylchloride, polyethylene, polyurethane. Polymeric materials suitable for use in forming nonwoven materials include polyolefins such as polyethylene and polypropylene, polyesters, nylons, ethylene vinyl acetate, ethylene methacrylate, copolymers of the above materials, and the like.

Pressure sensitive adhesives have been commonly found to work well for this purpose. The adhesive may be continuous or intermittent. For example, the adhesive may be applied in strips or across the entire surface of the backing layer. The adhesive may be applied via any suitable method, including, but not limited to spraying, printing, kiss coating, and direct slot coating.

### Garment-Facing Layer (Backing Layer)

Any conventional backing layer materials may be used, such as polyolefinic films or nonwoven webs. The backing layer may or may not be breathable. The backing layer may be suitable for printing.

### Cushioning Layer

Any conventional cushioning materials may be used within the device system. Suitable cushioning materials include comminuted wood pulp which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including co-form; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof.

### Proper Electrode Positioning and Shape

Typically, selectivity in activating nerves has required electrodes to be implanted surgically on or near nerves or, for transcutaneous stimulation, has required electrial beam steering. The device and system described herein, however, provides selective activation to the tibial nerve 300 transcutaneously, without the need for beam steering. The effectiveness of the device and system described herein for treating and/or managing the symptoms of OAB depends, in large part, on positioning the electrodes at the appropriate location on the user's body in order to stimulate the tibial nerve 300. Without proper positioning of the electrodes, the user may not get the full benefit or, in some cases, any benefit from the device.

The tibial nerve 300 is a branch of the sciatic nerve that passes alongside the tibia and into the foot. At the ankle, the tibial nerve 300 is relatively close to the surface of the skin. Positioning the electrodes at a location where the tibial nerve 300 is close to the skin allows for the device itself to be smaller and lighter, because less battery power is required to stimulate the nerve close to the skin. Also, there are few other (untargeted) nerves in the ankle, making it less likely that the generated electric field affects untargeted nerves.

The electrodes may be positioned on the ankle in a variety of ways, however, not all positions are effective. For example, it is not preferred to position one or more of the electrodes on the lateral side of the ankle, as the tibial nerve 300 is not close to the skin on the lateral side of the ankle. Also, positioning on the lateral side of the ankle may activate pain nerves. Positioning the electrodes above the ankle is not preferred, as the tibial nerve 300 is covered by muscle above the ankle, which requires more energy is required to activate the nerve. Positioning the electrodes on the top or bottom of the foot is not preferred, as the tibial nerve 300 branches out in the foot, making it more difficult to activate at this location (also such positioning may cause discomfort while walking or standing).

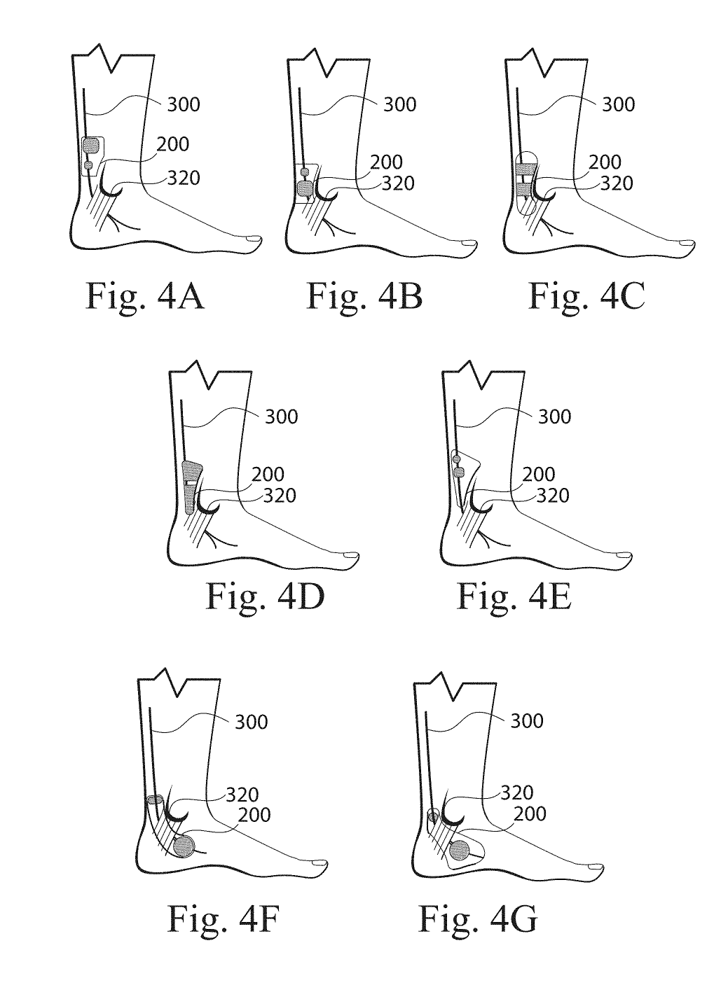

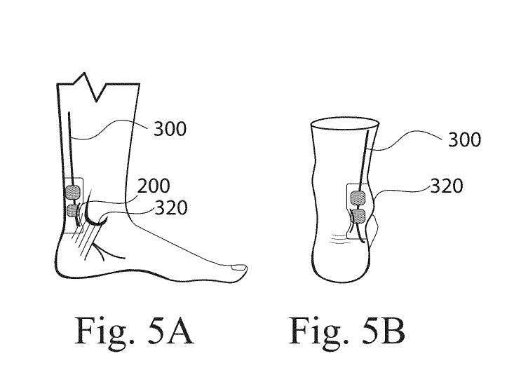

Also, the electrodes may be positioned transversely across the path of the tibial nerve 300 or axially along the path of the tibial nerve 300. If the electrodes are arranged transversely across the nerve, then more battery power may be required to stimulate the nerve. The electrodes are preferably arranged axially along the nerve (as shown in FIG. 4a-g, FIG. 5a-b). Preferably the electrodes 102 or the electrical field generated by the electrodes intersect or overlap the tibial nerve 300.

For a comparative example, a tibial nerve stimulation system, where at least four electrodes are placed around the circumference of the ankle, is known. This configuration may require more precise electrode placement, likely by a medical professional, and electrical beam steering technology to reliably stimulate the tibial nerve 300. This approach may also require significantly more electrical energy, e.g., higher voltage, which can negatively activate the pain nerves.

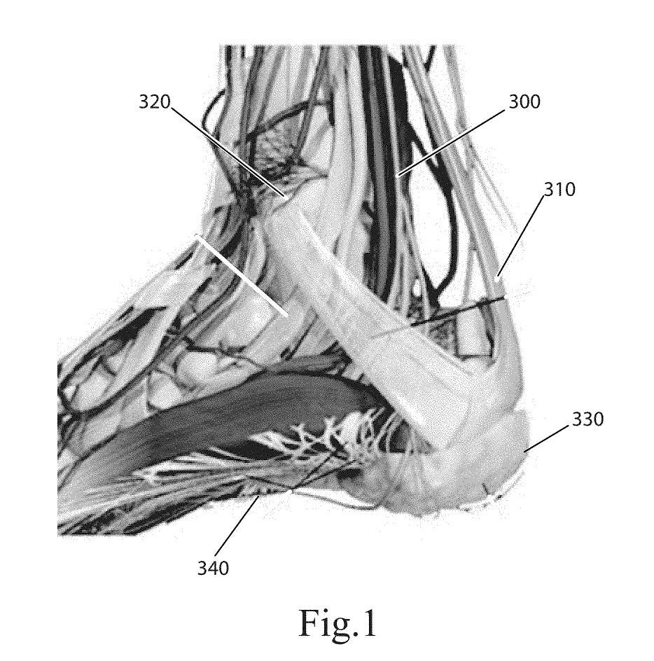

It has been found that communicating to the user to align and affix the device or system, such that the electrodes are positioned in the desired locations on the ankle, is a challenge. Instructions including writing, illustrations, or orientation marks for properly aligning and affixing the device or system may be included on the device or system itself, in the packaging for the device or system, or on the packaging of the device or system. However, users may dispose of the instructions without reading them, for example, when disposing of the packaging, or simply ignore the instructions. A very effective way to communicate to a user to properly affix the device or system, such that the electrodes are positioned in the desired location on the ankle, is by selecting one or more anatomical landmarks on the ankle and making the shape of the device or system complement or complete the anatomical landmark(s). By selecting an appropriate anatomical landmark(s) on the ankle and then by designing the shape of the device or system to complement or complete the landmark, a device or system that is intuitive for the user to align and affix properly is provided. Suitable anatomical landmarks include the achilles tendon (310), the heel 330, the arch (340), the medial malleolus (320), the lateral malleolus (not shown), or combinations thereof, as seen in FIG. 1. The arch (340) and/or the medial malleolus (320) are preferred. The medial malleolus (320) is most preferred. These anatomical landmarks on the ankle are prominent and fairly easy for the user to identify, by sight or touch.

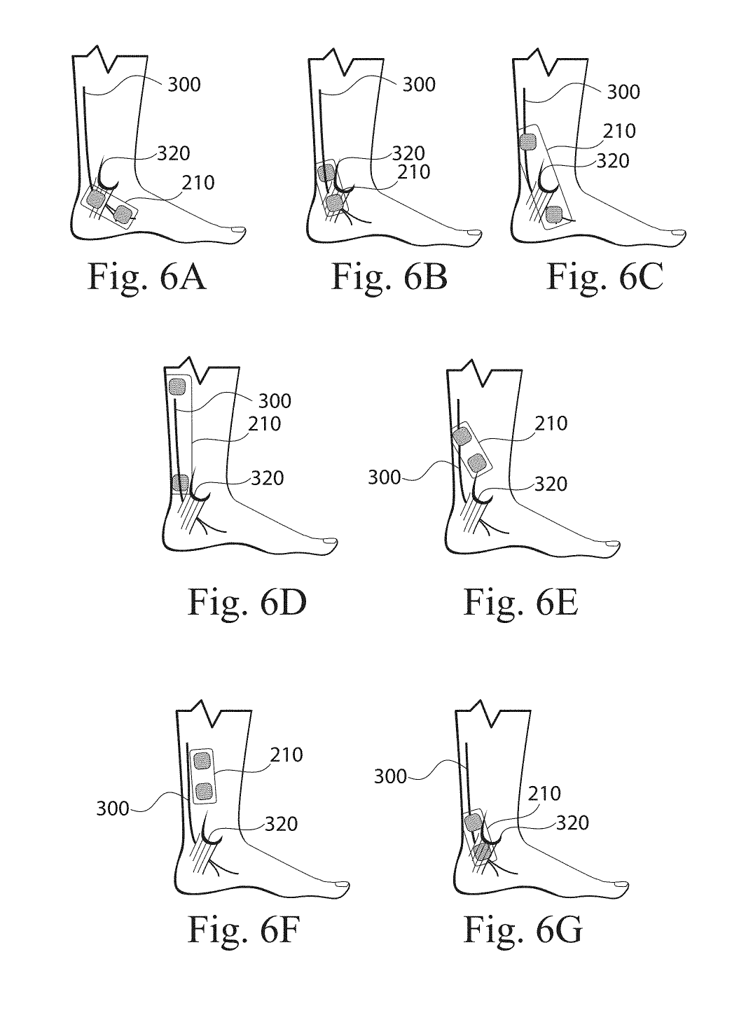

The sides and edges of the ankle are generally nonlinear. As used in the context of the present specification, the term “nonlinear” refers to any of various curved, as opposed to straight, lines. As shown in FIGS. 4a-g and FIGS. 5a-b, the devices and systems described herein may have at least one nonlinear edge (200), which may align with a nonlinear edge of one of the anatomical landmarks described herein, preferably the medial malleolus 320 (in FIGS. 4a-g and FIGS. 5a-b, not every nonlinear edge is labeled). The medial malleolus 320 is a circular protrusion on the medial side of the ankle. As used in the context of the present specification, a circle has one edge—its circumference. As shown in FIGS. 4a-g, the nonlinear edge (200) of the system may be concave relative to the medial malleolus (320) and posterior to the medial malleolus (320). That is to say, the nonlinear edge (200) of the system may curve around the posterior border (or posterior edge) of the medial malleolus (320). Examples of inventive systems are shown in FIGS. 4a-g and FIGS. 5a-b and examples of comparative systems are shown in FIGS. 6a-g. The edges (210) of the comparative systems are linear, though the corners may be rounded. As seen in FIGS. 6a-g, the linear edges (210) of the comparative systems do not align with the nonlinear edges of the medial malleolus (320), and, consequently, the electrodes of the comparative systems show in FIGS. 6a-g are not positioned to stimulate the tibial nerve 300 effectively. Because the comparative systems show in FIGS. 6a-g are more difficult for the user to align properly, the comparative systems, in the placements on the ankles shown in FIGS. 6a-g, may provide less benefit or no benefit at all, in terms of treating OAB and its symptoms.

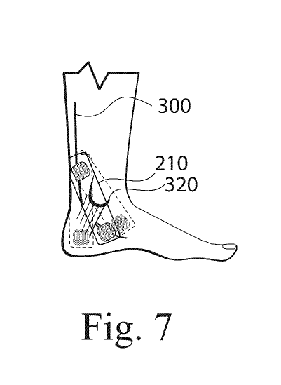

As shown in FIG. 7, when keeping one of the electrodes 102a at a fixed position, there are numerous possible placements of the comparative systems (three different placements are shown in FIG. 7). The electrodes are properly positioned in only one of the three placements shown in FIG. 7 (device with black borders and electrodes versus devices with grey borders and electrodes). Thus, the probability of improperly placing the comparative systems may be greater, as compared to the inventive systems. In order to correct for or compensate for the higher probability of improper placement, the comparative systems may require larger electrodes and more battery power to stimulate the tibial nerve 300, which makes the devices more obtrusive and less discrete.

### Dimensions

The device or system may weigh less than about 30 g, preferably from about 1 g to about 30 g, more preferably from about 5 g to about 20 g, even more preferably from about 2 g to about 15 g. The area of the body-facing surface of the device or system may range from about 500 mm² to about 9000 mm², preferably about 750 mm² to about 4100 mm², more preferably about 750 mm² to about 3500 mm². The ratio of the body-facing surface area of the device or system to the sum of the body-facing surface areas of the electrodes may be from about 1:1 to about 5:1, preferably about 1:1 to about 4:1, more preferably about 1:1 to about 3:1. The device or system may have a maximum thickness of about 0.10 mm to about 15 mm, preferably about 0.10 mm to about 10 mm, more preferably 0.10 mm to about 5 mm Thickness may be measured using any number of known methods.

### Camouflaging

The topical nerve activation system described herein may be camouflaged to make the system less obtrusive and more discrete. Camouflaging includes matching the color of the system to the skin tone of the user, matching the color of the system to a user's garment, or disguising the system as something less upsetting to the user, such as a blister pad. Camouflaging may also include a very colorful, bright system or a system having a colorful design, to make the system look like an accessory or a tattoo.

### Method of Treating OAB

The present disclosure also relates to a method of treating overactive bladder and/or its symptoms comprising: a) providing the device or system as disclosed herein to a user experiencing a symptom of overactive bladder; b) affixing the device or system as disclosed herein to one of the user's medial ankles, preferably proximate to and posterior to the medial malleolus; c) optionally turning on the device or system of any one of the preceding paragraphs; d) activating or stimulating the tibial nerve of the user.

### Combinations

A. A topical nerve stimulation device for treating overactive bladder in a human user, the device comprising:
a. at least two electrodes 102, preferably a positive electrode and a negative electrode, configured to contact a surface of the medial ankle;
b. a power source 112 in electrical communication with the electrodes;
wherein the device is shaped and configured for placement proximate to the medial malleolus 320, preferably proximate and posterior to the medial malleolus 320, to stimulate the tibial nerve 300.
   B. A topical nerve stimulation system for treating overactive bladder in a human user, the system comprising:
(a) a body-facing layer 510;
(b) a garment-facing layer 520;
   (c) at least two electrodes 102, preferably a positive electrode and a negative electrode, the electrodes configured to contact a surface of the medial ankle, and a power source 112 in electrical communication with the electrodes 102, the power source 112 disposed intermediate the garment-facing layer 520 and the body-facing layer 510;
   wherein the system is shaped and configured for placement proximate to the medial malleolus 320, preferably proximate and posterior to the medial malleolus 320, to stimulate the tibial nerve 300.
   C. The topical nerve stimulation system according to paragraph B, wherein an adhesive is disposed on the body-facing layer 510.
   D. The topical nerve stimulation system according to paragraph C, wherein a removable liner is disposed on the adhesive prior to use.
   E. The topical nerve stimulation system of paragraph D, wherein removal of the liner activates the power source 112.
   F. The topical nerve stimulation system of paragraph B, wherein the electrodes 102 are integrated into the body-facing layer 510.
   G. The topical nerve stimulation system of paragraph B, wherein the system further comprises a cushioning layer 530 disposed intermediate the body-facing layer 510 and the garment-facing layer 520.
   H. The topical nerve stimulation device of paragraph B, wherein the body-facing layer 510 and the garment-facing layer 520 are bonded together.
   I. The topical nerve stimulation device of paragraph B, wherein the body-facing layer 510 comprises a nonwoven material, a plastic material, a latex material, or a mixture thereof.
   J. The topical nerve stimulation device of paragraph B, wherein the garment-facing layer 520 comprises coated paper, a nonporous film, a porous film, a woven material, a non-woven fibrous material, or combinations thereof.
   K. A method of treating overactive bladder and/or its symptoms comprising:
(a) providing the device or system of any one of the preceding paragraphs to a user experiencing a symptom of overactive bladder;
(b) affixing the device or system of any one of the preceding paragraphs to one of the user's medial ankles, preferably proximate to and posterior to the medial malleolus 320;
(c) optionally turning on the device or system of any one of the preceding paragraphs;
(d) activating or stimulating the tibial nerve 300 of the user.
   L. The topical nerve stimulation device or system according to any one of the preceding paragraphs, wherein the device or system is disposable.
   M. The topical nerve stimulation device or system according to any one of the preceding paragraphs, wherein the device or system does not contact or travel the circumference of the ankle.
   N. The topical nerve stimulation device or system according to any one of the preceding paragraphs, wherein the device or system further comprises an electrical signal generator 106 in electrical communication with the at least two electrodes 102 and a signal activator 108 in electrical communication with the electrical signal generator 106, wherein the power source 112 is in electrical communication with the electrical signal generator 106 and the signal activator 108.
   O. The topical nerve stimulation device or system according to any one of the preceding paragraphs, wherein the device or system further comprises a signal receiver 114, preferably an antenna, in electrical communication with the electrical signal generator 106.
   P. The topical nerve stimulation device or system according to any one of the preceding paragraphs, wherein the electrodes 102 are configured to be positioned axially along the path of the tibial nerve 300, preferably the electrodes 102 or the electrical field generated by the electrodes intersect the tibial nerve 300.
   Q. The topical nerve stimulation device or system according to any one of the preceding paragraphs, wherein none of the electrodes 102 are configured to be positioned on the top of the foot, on the sole of the foot, or on the lateral ankle.
   R. The topical nerve stimulation device or system according to any one of the preceding paragraphs, wherein the device or system comprises two electrodes 102.
   S. The topical nerve stimulation device or system according to any one of the preceding paragraphs, wherein the device or system comprises at least one nonlinear edge 200.
   T. The topical nerve stimulation device or system according to any one of the preceding paragraphs, wherein the nonlinear edge 200 of the device or system is alignable with the medial malleolus 320.
   U. The topical nerve stimulation device or system according to any one of the preceding paragraphs, wherein the nonlinear edge 200 of the device or system is concave relative to and posterior to the medial malleolus 320.
   V. The topical nerve stimulation device or system according to any one of the preceding paragraphs, wherein the nonlinear edge 200 of the device or system curves around the medial malleolus 320, preferably posterior to the medial malleolus 320.
   W. The topical nerve stimulation device or system according to any one of the preceding paragraphs, wherein the area of the body-facing surface of each electrode 102 is from about 500 mm² to about 1100 mm², wherein the electrodes 102 may have different or identical body-facing surface areas.
   X. The topical nerve stimulation device or system according to any one of the preceding paragraphs, wherein the sum of the body-facing surface areas of the electrodes 102 is less than 3,000 mm², preferably from about 1050 mm² to about 2100 mm².
   Y. The topical nerve stimulation device or system according to any one of the preceding paragraphs, wherein the body-facing surface area of the device or system is from about 500 mm² to about 9000 mm², preferably about 750 mm² to about 4100 mm², more preferably about 750 mm² to about 3500 mm².
   Z. The topical nerve stimulation device or system according to any one of the preceding paragraphs, wherein the ratio of the body-facing surface area of the device or system to the sum of the body-facing surface areas of the electrodes 102 is from about 1:1 to about 5:1, preferably about 1:1 to about 3:1, more preferably about 1:1 to about 2:1.
   AA. The topical nerve stimulation device or system according to any one of the preceding paragraphs, wherein the maximum thickness of the device or system is about 0.10 mm to about 15 mm, preferably about 0.10 mm to about 10 mm, more preferably about 0.10 mm to about 5 mm.
   BB. The topical nerve stimulation device or system according to any one of the preceding paragraphs, wherein the device or system weighs from about 1 g to about 30 g, preferably from about 5 g to about 20 g, more preferably from about 2 g to about 15 g.
   CC. The topical nerve stimulation device or system according to any one of the preceding paragraphs, wherein the power source 112 is a battery.
   DD. The topical nerve stimulation device or system according to paragraph CC, wherein the capacity of the battery 112 is less than about 1000 mAh, preferably from about 1 mAh to about 500 mAh, more preferably about 1 mAh to about 100 mAh, even more preferably from about 1 to about 50 mAh.
   EE. The topical nerve stimulation device or system according to any one of the preceding paragraphs, wherein the edge-to-edge distance between the electrodes 102 is about 1 mm to abot 100 mm, preferably about 5 mm to about 80 mm, more preferably about 10 mm to about 60 mm.
   FF. The topical nerve stimulation device or system according to any one of the preceding paragraphs, wherein the positive electrode(s) is placed distal to the negative electrode.

This application claims the benefit of U.S. Provisional Application Ser. No. 62/574,625, the entirety of which is incorporated by reference herein. Also, numerous suitable nerve stimulation devices, including components of the devices and protocols for operating the devices, are described in U.S. patent application Ser. No. 15/912,058, and U.S. Pat. No. 10,016,600, both of which are hereby incorporated by reference.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical nerve stimulation device for treating overactive bladder in a human user, the device comprising:
c. at least two electrode configured to contact a surface of the medial ankle;
d. a power source in electrical communication with the electrodes;
wherein the device is shaped and configured for placement proximate to the medial malleolus to stimulate the tibial nerve.

2. A topical nerve stimulation system for treating overactive bladder in a human user, the system comprising:
(a) a body-facing layer;
(b) a garment-facing layer;
(c) at least two electrodes, the electrodes configured to contact a surface of the medial ankle, and a power source in electrical communication with the electrodes, the power source disposed intermediate the garment-facing layer and the body-facing layer;
wherein the system is shaped and configured for placement proximate to the medial malleolus to stimulate the tibial nerve.

3. The topical nerve stimulation system according to claim 2, wherein an adhesive is disposed on the body-facing layer.

4. A method of treating overactive bladder and/or its symptoms comprising:
(e) providing the device of claim 1 to a user experiencing a symptom of overactive bladder;
(f) affixing the device of claim 1 to one of the user's medial ankles;
(g) activating or stimulating the tibial nerve of the user.

5. The topical nerve stimulation device of claim 1, wherein the device is disposable.

6. The topical nerve stimulation device of claim 1, wherein the device does not travel the circumference of the ankle.

7. The topical nerve stimulation device of claim 1, wherein the device further comprises an electrical signal generator in electrical communication with the at least two electrodes and a signal activator in electrical communication with the electrical signal generator, wherein the power source is in electrical communication with the electrical signal generator and the signal activator.

8. The topical nerve stimulation device of claim 1, wherein the device further comprises a signal receiver in electrical communication with the electrical signal generator.

9. The topical nerve stimulation device of claim 1, wherein the electrodes are configured to be positioned axially along the path of the tibial nerve.

10. The topical nerve stimulation device of claim 1, wherein none of the electrodes are configured to be positioned on the top of the foot, on the sole of the foot, or on the lateral ankle.

11. The topical nerve stimulation device of claim 1, wherein the device comprises two electrodes.

12. The topical nerve stimulation device of claim 1, wherein the device comprises at least one nonlinear edge.

13. The topical nerve stimulation device of claim 12, wherein the nonlinear edge of the device is alignable with the medial malleolus.

14. The topical nerve stimulation device of claim 12, wherein the nonlinear edge of the device is concave relative to and posterior to the medial malleolus.

15. The topical nerve stimulation device of claim 12, wherein the nonlinear edge of the device curves around the medial malleolus.

16. The topical nerve stimulation device of claim 1, wherein the area of the body-facing surface of each electrode is from about 500 mm² to about 1100 mm², wherein the electrodes may have different or identical body-facing surface areas.

17. The topical nerve stimulation device of claim 1, wherein the sum of the body-facing surface areas of the electrodes is less than 3,000 mm².

18. The topical nerve stimulation device of claim 1, wherein the body-facing surface area of the device is from about 500 mm² to about 9000 mm².

19. The topical nerve stimulation device of claim 1, wherein the ratio of the body-facing surface area of the device to the sum of the body-facing surface areas of the electrodes is from about 1:1 to about 5:1.

20. The topical nerve stimulation device of claim 1, wherein the maximum thickness of the device is about 0.10 mm to about 15 mm.

21. The topical nerve stimulation device of claim 1, wherein the device weighs from about 1 g to about 30 g.

22. The topical nerve stimulation device of claim 1, wherein the power source is a battery.

23. The topical nerve stimulation device of claim 24, wherein the capacity of the battery is about 1 mAh to about 100 mAh.

24. The topical nerve stimulation device of claim 1, wherein the edge-to-edge distance between the electrodes is about 1 mm to about 100 mm.
